(19) United States
(12) Patent Application Publication
(10) Pub. No.: US 2004/0191797 A1
(43) Pub. Date: Sep. 30, 2004

(54) Human G-protein coupled receptor

(75) Inventors: Willy Deleersnijder, Weesp (NL); Guy Nys, Weesp (NL); Fan Zhang, Weesp (NL)

Correspondence Address: FINNEGAN, HENDERSON, FARABOW, GARRETT & DUNNER, LLP, 1300 I STREET, NW, WASHINGTON, DC, 20005, US

(73) Assignee: Solvay Pharmaceuticals B.V.

(21) Appl. No.: 10/608,990

(22) Filed: Dec. 10, 2003

Related U.S. Application Data
(62) Division of application No. 10/030,459, filed on Jun. 5, 2002, now Pat. No. 6,702,269.

(30) Foreign Application Priority Data
Jul. 17, 2000 (WO) PCT/EP00/06878
Jul. 15, 1999 (EP) 99202326.7
Jul. 15, 1999 (NL) 1012611

(51) Int. Cl. C12Q 1/68; C07H 21/04; C07K 14/705

(52) U.S. Cl. 435/6; 435/69.1; 435/320.1; 435/325; 530/350; 530/388.22; 536/23.5

(57) ABSTRACT

The present invention relates to novel identified polynucleotides, polypeptides encoded by them and to the use of such polynucleotides and polypeptides, and to their production. More particularly, the polynucleotides and polypeptides of the present invention relate to the G-protein coupled receptor family, referred to as IGS1-family. The invention also relates to inhibiting or activating the action of such polynucleotides and polypeptides, to a vector containing said polynucleotides, a host cell containing such vector and transgenic animals where the IGS1-gene is either overexpressed, misexpressed, underexpressed or suppressed (knock-out animals). The invention further relates to a method for screening compounds capable to act as an agonist or an antagonist of said G-protein coupled receptor family IGS1 and the use of IGS1 polypeptides and polynucleotides and agonists or antagonists to the IGS1 receptor family in the treatment of psychiatric and CNS disorders.

The present invention relates to novel identified polynucleotides, polypeptides encoded by them and to the use of such polynucleotides and polypeptides, and to their production. More particularly, the polynucleotides and polypeptides of the present invention relate to a G-protein coupled receptor (GPCR), hereinafter referred to as IGS1. The invention also relates to inhibiting or activating the action of such polynucleotides and polypeptides, to a vector containing said polynucleotides, a host cell containing such vector and transgenic animals where the IGS1-gene is either overexpressed, misexpressed, underexpressed and/or suppressed (knock-out animals). The invention further relates to a method for screening compounds capable to act as an agonist or an antagonist of said G-protein coupled receptor IGS1.

### BACKGROUND OF THE INVENTION

It is well established that many medically significant biological processes are mediated by proteins participating in signal transduction pathways that involve G-proteins and/or second messengers; e.g., cAMP (Lefkowitz, Nature, 1991, 351:353-354). Herein these proteins are referred to as proteins participating in pathways with G-proteins. Some examples of these proteins include the GPC receptors, such as those for adrenergic agents and dopamine (Kobilka, B. K., et al., Proc. Natl. Acad. Sci., USA, 1987, 84:46-50; Kobilka, B. K., et al., Science, 1987, 238:650-656; Bunzow, J. R., et al., Nature, 1988, 336:783-787), G-proteins themselves, effector proteins, e.g., phospholipase C, adenylate cyclase, and phosphodiesterase, and actuator proteins, e.g., protein kinase A and protein kinase C (Simon, M. I., et al., Science, 1991, 252:802-8).

For example, in one form of signal transduction, upon hormone binding to a GPCR the receptor interacts with the heterotrimeric G-protein and induces the dissociation of GDP from the guanine nucleotide-binding site. At normal cellular concentrations of guanine nucleotides, GTP fills the site immediately. Binding of GTP to the α subunit of the G-protein causes the dissociation of the G-protein from the receptor and the dissociation of the G-protein into α and βγ subunits. The GTP-carrying form then binds to activated adenylate cyclase. Hydrolysis of GTP to GDP, catalyzed by the G-protein itself (α subunit possesses an intrinsic GTPase activity), returns the G-protein to its basal, inactive form. The GTPase activity of the α subunit is, in essence, an internal clock that controls an on/off switch. The GDP bound form of the α subunit has high affinity for βγ and subsequent reassociation of αGDP with βγ returns the system to the basal state. Thus the G-protein serves a dual role, as an intermediate that relays the signal from receptor to effector (in this example adenylate cyclase), and as a clock that controls the duration of the signal.

The membrane bound superfamily of G-protein coupled receptors has been characterized as having seven putative transmembrane domains. The domains are believed to represent transmembrane α-helices connected by extracellular or cytoplasmic loops. G-protein coupled receptors include a wide range of biologically active receptors, such as hormone, viral, growth factor and neuroreceptors.

The G-protein coupled receptor family includes dopamine receptors which bind to neuroleptic drugs used for treating CNS disorders. Other examples of members of this family include, but are not limited to calcitonin, adrenergic, neuropeptideY, somastotatin, neurotensin, neurokinin, capsaicin, VIP, CGRP, CRF, CCK, bradykinin, galanin, motilin, nociceptin, endothelin, cAMP, adenosine, muscarinic, acetylcholine, serotonin, histamine, thrombin, kinin, follicle stimulating hormone, opsin, endothelial differentiation gene-1, rhodopsin, odorant, and cytomegalovirus receptors.

Most G-protein coupled receptors have single conserved cysteine residues in each of the first two extracellular loops which form disulfide bonds that are believed to stabilize functional protein structures. The 7 transmembrane regions are designated as TM1, TM2, TM3, TM4, TM5, TM6 and TM7. The cytoplasmic loop which connects TM5 and TM6 may be a major component of the G-protein binding domain.

Most G-protein coupled receptors contain potential phosphorylation sites within the third cytoplasmic loop and/or the carboxy terminus. For several G-protein coupled receptors, such as the β-adrenoreceptor, phosphorylation by protein kinase A and/or specific receptor kinases mediates receptor desensitization.

Recently, it was discovered that certain GPCRs, like the calcitonin-receptor like receptor, might interact with small single pass membrane proteins called receptor activity modifying proteins (RAMP's). This interaction of the GPCR with a certain RAMP is determining which natural ligands have relevant affinity for the GPCR-RAMP combination and regulate the functional signaling activity of the complex (McLathie, L. M. et al., Nature (1998) 393:333-339).

For some receptors, the ligand binding sites of G-protein coupled receptors are believed to comprise hydrophilic sockets formed by several G-protein coupled receptor transmembrane domains, said sockets being surrounded by hydrophobic residues of the G-protein coupled receptors. The hydrophilic side of each G-protein coupled receptor transmembrane helix is postulated to face inward and form a polar ligand-binding site. TM3 has been implicated in several G-protein coupled receptors as having a ligand-binding site, such as the TM3 aspartate residue. TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 1989, 10:317-331). Different G-protein a-subunits preferentially stimulate particular effectors to modulate various biological functions in a cell. Phosphorylation of cytoplasmic residues of G-protein coupled receptors has been identified as an important mechanism for the regulation of G-protein coupling of some G-protein coupled receptors. G-protein coupled receptors are found in numerous sites within a mammalian host.

Receptors—primarily the GPCR class—have led to more than half of the currently known drugs (Drews, Nature Biotechnology, 1996, 14: 1516). This indicates that these receptors have an established, proven history as therapeutic targets. The new IGS1 GPCR described in this invention clearly satisfies a need in the art for identification and characterization of further receptors that can play a role in diagnosing, preventing, ameliorating or correcting dysfunctions, disorders, or diseases, hereafter generally referred to as “the Diseases”. The Diseases include, but are not limited to, psychiatric and CNS disorders, including schizophrenia, episodic paroxysmal anxiety (EPA) disorders such as obsessive compulsive disorder (OCD), post traumatic stress disorder (PTSD), phobia and panic, major depressive disorder, bipolar disorder, Parkinson's disease, general anxiety disorder, autism, delirium, multiple sclerosis, Alzheimer disease/dementia and other neurodegenerative diseases, severe mental retardation, dyskinesias, Huntington's disease, Tourett's syndrome, tics, tremor, dystonia, spasms, anorexia, bulimia, stroke, addiction/dependency/craving, sleep disorder, epilepsy, migraine; attention deficit/hyperactivity disorder (ADHD); cardiovascular diseases, including heart failure, angina pectoris, arrhythmias, myocardial infarction, cardiac hypertrophy, hypotension, hypertension—e.g. essential hypertension, renal hypertension, or pulmonary hypertension, thrombosis, arteriosclerosis, cerebral vasospasm, subarachnoid hemorrhage, cerebral ischemia, cerebral infarction, peripheral vascular disease, Raynaud's disease, kidney disease—e.g. renal failure; dyslipidemias; obesity; emesis; gastrointestinal disorders, including irritable bowel syndrome (IBS), inflammatory bowel disease (IBD), gastroesophagal reflux disease (GERD), motility disorders and conditions of delayed gastric emptying, such as post operative or diabetic gastroparesis, and diabetes, ulcers—e.g. gastric ulcer; diarrhoea; other diseases including osteoporosis; inflammations; infections such as bacterial, fungal, protozoan and viral infections, particularly infections caused by HIV-1 or HIV-2; pain; cancers; chemotherapy induced injury; tumor invasion; immune disorders; urinary retention; asthma; allergies; arthritis; benign prostatic hypertrophy; endotoxin shock; sepsis; complication of diabetes mellitus; and gynaecological disorders.

In particular, the new IGS1 GPCR described in this invention satisfies a need in the art for identification and characterization of further receptors that can play an important role in diagnosing, preventing, ameliorating or correcting psychiatric and CNS dysfunctions, disorders, or diseases, especially movement dysfunctions, disorders, or diseases,.such as tics, tremor, Tourette's syndrome, Parkinson's disease, Huntington's disease, dyskinesias, dystonia and spasms.

### SUMMARY OF THE INVENTION

In one aspect, the invention relates to IGS1 polypeptides and recombinant materials and methods for their production. Another aspect of the invention relates to methods for using such IGS1 polypeptides and polynucleotides. Such uses include, but are not limited to, treatment of one of the Diseases as mentioned above. In particular the uses include treatment of psychiatric and CNS disorders, especially movement disorders, such as tics, tremor, Tourette's syndrome, Parkinson's disease, Huntington's disease, dyskinesias, dystonia and spasms.

In still another aspect, the invention relates to methods to identify agonists and antagonists using the materials, provided by the invention, and treating conditions associated with IGS1 imbalance with the identified compounds. Yet another aspect of the invention relates to diagnostic assays for detecting diseases associated with inappropriate IGS1 activity or levels. A further aspect of the invention relates to animal-based systems which act as models for disorders arising from aberrant expression or activity of IGS1.

### BRIEF DESCRIPTION OF THE FIGURES

FIG. 1 Schematic representation of the relative positions of the different clones that were isolated to generate the consensus IGS1 contig. PCR primers used for cloning are indicated (IP#). Clone HB4693 was obtained via overlap PCR of clones HNT1398 and HNT1413. The position of the IGS1 coding sequence (IGS1PROT) is indicated with asterisks(“*”). The location of EST20889 and EST accession n° A1672141 is indicated with “==” and the location of the IGS1 DNA sequence with “++”. IGS1 DNA is the part of the IGS1 contig of which the sequence was determined on at least 4 independent cDNA clones at every hucleotide position.

FIG. 2. Multiple tissue expression array analysis using a human IGS1 probe. There are a lot of spurious signals on this membrane. Only the signals indicated by the arrows do coincide with the position of the deposited RNA and are specific.

FIG. 3. Northern blot analysis of human IGS1 on RNA from different brain regions.

TABLE 1
IGS1-DNA of SEQ ID NO: 1

5′-
GCCTGCAACCTGTCYCACGCCCTCTGGCTGTTGCCATGACGTCCACCTGCA
CCAACAGCACGCGCGAGAGTAACAGCAGCCACACGTGCATGCCCCTCTCCA
AAATGCCCATCAGCCTGGCCCACGGCATCATCCGCTCAACCGTGCTGGTTA
TCTTCCTCGCCGCCTCTTTCGTCGGCAACATAGTGCTGGCGCTAGTGTTGC
AGCGCAAGCCGCAGCTGCTGCAGGTGACCAAGCGTTTTATCTTTAACCTCCT
CGTCACCGACCTGCTGCAGATTTGGCTCGTGGCCCCCTGGGTGGTGGCCA
CCTCTGTGCCTCTCTTCTGGCCCCTCAACAGCCACTTCTGCACGGCCCTGG
TTAGCCTCACCCACCTGTTCGCCTTCGCCAGCGTCAACACCATTGTCTTGGT
GTCAGTGGATCGCTACTTGTCCATCATCCACCCTCTCTCGTACCCGTCCAAG
ATGACCCAGCGCCGCGGTTACCTGCTCCTCTATGGCACGTGGATTGTGGCC
ATCCTGCAGAGCACTCCTCCACTCTACGGCTGGGGCCAGGCTGCCTTTGAT
GAGCGCAATGCTCTCTGCTCCATGATCTGGGGGGCCAGCCGCAGCTACACT
ATTCTCAGCGTGGTGTCCTTCATCGTCATTCCACTGATTGTCATGATTGCCT
GCTACTCCGTGGTGTTCTGTGCAGCCCGGAGGCAGCATGCTCTGCTGTACA
ATGTCAAGAGACACAGCTTGGAAGTGCGAGTCAAGGACTGTGTGGAGAATG
AGGATGAAGAGGGAGCAGAGAAGAAGGAGGAGTTCCAGGATGAGAGTGAG
TTTCGCCGCCAGCATGAAGGTGAGGTCAAGGCCAAGGAGGGCAGAATGGA
AGCCAAGGACGGCAGCCTGAAGGCCAAGGAAGGAAGCACGGGGACCAGTG
AGAGTAGTGTAGAGGCCAGGGGCAGCGAGGAGGTCAGAGAGAGCAGCACG
GTGGCCAGCGACGGCAGCATGGAGGGTAAGGAAGGCAGCACCAAAGTTGA
GGAGAACAGCATGAAGGCAGACAAGGGTCGCACAGAGGTCAACCAGTGCA
GCATTGACTTGGGTGAAGATGGCATGGAGTTTGGTGAAGACGACATCAATTT
CAGTGAGGATGACGTCGAGGCAGTGAACATCCCGGAGAGCCTCCCACCCA
GTCGTCGTAACAGCAACAGCAACCCTCCTCTGCCCAGGTGCTACCAGTGCA
AAGCTGCTAAAGTGATCTTCATCATCATTTTCTCCTATGTGCTATCCCTGGGG
CCCTACTGCTTTTTAGCAGTCCTGGCCGTGTGGGTGGATGTCGAAACCGAG
GTACCCCAGTGGGTGATCACCATAATCATCTGGCTTTTCTTCCTGCAGTGCT
GCATCCACCCCTATGTCTATGGCTACATGCACAAGACCATTAAGAAGGAAAT
CCAGGACATGCTGAAGAAGTTCTTCTGCAAGGAAAAGCCCCCGAAAGAAGA
TAGCCACCCAGACCTGCCCGGAACAGAGGGTGGGACTGAAGGCAAGATTG
TCCCTTCCTACGATTCTGCTACTTTTCCTTGAAGTTAGTTCTAAGGCAAACCT
TGAAAATCAGTCCTTCAGCCACAGCTATTTAGAGCTTTAAAACTACCAGGTTC
AATCACTGGTTATGCTTTCTGTG-3′

TABLE 2
IGSI-protein of SEQ ID NO: 2

MTSTCTNSTRESNSSHTCMPLSKMPISLAHGIIRSTVLV
IFLAASFVGNIVLALVLQRKPQLLQVTNRFIFNLLVTDLL
QISLVAPWVVATSVPLFWPLNSHFCTALVSLTHLFAFAS
VNTIVLVSVDRYLSIIHPLSYPSKMTQRRGYLLLYGTWI
VAILQSTPPLYGWGQAAFDERNALCSMIWGASPSYTIL
SVVSFIVIPLIVMIACYSVVFCAARRQHALLYNVKRHSL
EVRVKDCVENEDEEGAEKKEEFQDESEFRRQHEGEVK
AKEGRMEAKDGSLKAKEGSTGTSESSVEARGSEEVRE
SSTVASDGSMEGKEGSTKVEENSMKADKGRTEVNQCS
IDLGEDGMEFGEDDINFSEDDVEAVNIPESLPPSRRNS
NSNPPLPRCYQCKAAKVIFIIIFSYVLSLGPYCFLAVLAV
WVDVETQVPQWVITIIIWLFFLQCCIHPYVYGYMHKTIK
KEIQDMLKKFFCKEKPPKEDSHPDLPGTEGGTEGKIVP
SYDSATFP

### DESCRIPTION OF THE INVENTION

Structural similarity, in the context of sequences and motifs, exists among the IGS1 GPCR of the invention and other human GPCR's. In addition, IGS1 is expressed in brain tissues, in particular in caudate nucleus and putamen. Therefore, IGS1 is implied to play a role among other things in the Diseases mentioned above. IGS1 in particular is implied to play a role in psychiatric and CNS disorders, especially movement disorders, such as tics, tremor, Tourette's syndrome, Parkinson's disease, Huntington's disease, dyskinesias, dystonia and spasms.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited in this specification are herein incorporated by reference as if each individual publication were specifically and individually indicated to be incorporated by reference herein as though fully set forth.

Definitions

The following definitions are provided to facilitate understanding of certain terms used frequently herein.

“IGS1” refers, among others, to a polypeptide comprising the amino acid sequence set forth in SEQ ID NO:2, or an allelic variant thereof.

“Receptor Activity” or “Biological Activity of the Receptor” refers to the metabolic or physiologic function of said IGS1 including similar activities or improved activities or these activities with decreased undesirable side effects. Also included are antigenic and immunogenic activities of said IGS1.

“IGS1-gene” refers to a polynucleotide comprising the nucleotide sequence set forth in SEQ ID NO:1 or allelic variants thereof and/or their complements.

“Antibodies” as used herein includes polyclonal and monoclonal antibodies, chimeric, single chain, and humanized antibodies, as well as Fab fragments, including the products of a Fab or other immunoglobulin expression library.

“Isolated” means altered “by the hand of man” from the natural state and/or separated from the natural environment. Thus, if an “isolated” composition or substance that occurs in nature has been “isolated”, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single-and double-stranded regions, hybrid molecules comprising DNA and RNA that may be single-stranded or, more typically, double-stranded or a mixture of single- and double-stranded regions. In addition, “polynucleotide” may also include triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to short chains, commonly referred to as peptides, oligopeptides or oligomers, and to longer chains, generally referred to as proteins, and/or to combinations thereof. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well-described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol; cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth. Enzymol. (1990) 182:626-646 and Rattan et al., “Protein Synthesis: Posttranslational Modifications and Aging”, Ann. NY Acad. Sci. (1992) 663:48-62.

“Variant” as the term is used herein, is a polynucleotide or polypeptide that differs from a reference polynucleotide or polypeptide respectively, but retains essential properties such as essential biological, structural, regulatory or biochemical properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” is a measure of the identity of ndcleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed.; Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known .to skilled artisans (Carillo, H., and Lipton, D., SIAM J. Applied Math. (1988) 48:1073). Methods commonly employed to determine identity or similarity between two sequences include, but are not limited to, those disclosed in Guide to Huge Computers, Martin J. Bishop, ed., Academic Press, San Diego, 1994, and Carillo, H., and Lipton, D., SIAM J. Applied Math. (1988) 48:1073. Methods to determine identity and similarity are codified in computer programs. Preferred computer program methods to determine identity and similarity between two sequences include, but are not limited to, GCG program package (Devereux, J., et al., Nucleic Acids Research (1984) 12(1):387), BLASTP, BLASTN, FASTA (Atschul, S. F. et al., J. Molec. Biol. (1990) 215:403). The word “homology” may substitute for the words “identity”.

As an illustration, by a polynucleotide having a nucleotide sequence having at least, for example, 95% “identity” to a reference nucleotide sequence of SEQ ID NO: 1 is intended that the nucleotide sequence of the polynucleotide is identical to the reference sequence except that the polynucleotide sequence may include up to five nucleotide differences per each 100 nucleotides of the reference nucleotide sequence of SEQ ID NO: 1. In other words, to obtain a polynucleotide having a nucleotide sequence at least 95% identical to a reference nucleotide sequence, up to any 5% of the nucleotides in the reference sequence may be deleted or substituted with another nucleotide, or a number of nucleotides up to any 5% of the total nucleotides in the reference sequence may be inserted into the reference sequence, or in a number of nucleotides of up to any 5% of the total nucleotides in the reference sequence there may be a combination of deletion, insertion and substitution. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO:2 is intended that the amino acid sequence of the polypeptide is identical to the reference sequence except that the polypeptide sequence may include up to five amino acid alterations per each 100 amino acids of the reference amino acid of SEQ ID NO: 2. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

Polypeptides of the Invention

In one aspect, the present invention relates to IGS1 polypeptides (including IGS1 proteins). The IGS1 polypeptides include the polypeptide of SEQ ID NO:2 and the polypeptide having the amino acid sequence encoded by the DNA insert contained in the deposit no. CBS 102049, deposited on Jul. 15, 1999 at the Centraalbureau voor Schimmelcultures at Baarn the Netherlands; as well as polypeptides comprising the amino acid sequence of SEQ ID NO:2 and the polypeptide having the amino acid sequence encoded by the DNA insert contained in the deposit no. CBS 102049 at the Centraalbureau voor Schimmelcultures at Baarn the Netherlands and polypeptides comprising the amino acid sequence which have at least 80% identity to that of SEQ ID NO:2 and/or the polypeptide having the amino acid sequence encoded by the DNA insert contained in the deposit no. CBS 102049 at the Centraalbureau voor Schimmelcultures at Baarn the Netherlands over its entire length, and still more preferably at least 90% identity, and even still more preferably at least 95% identity to said amino acid sequence. Furthermore, those with at least 97%, in particular at least 99%, are highly preferred. Also included within IGS1 polypeptides are polypeptides having the amino acid sequence which have at least 80% identity to the polypeptide having the amino acid sequence of SEQ ID NO: 2 or the polypeptide having the amino acid sequence encoded by the DNA insert contained in the deposit no. CBS 102049 at the Centraalbureau voor Schimmelcultures at Baarn the Netherlands over its entire length, and still more preferably at least 90% identity, and even still more preferably at least 95% identity to SEQ ID NO: 2. Furthermore, those with at least 97%, in particular at least 99% are highly preferred. Preferably IGS1 polypeptides exhibit at least one biological activity of the receptor.

The IGS1 polypeptides may be in the form of the “mature” protein or may be a part of a larger protein such as-a fusion protein. It is often advantageous to include an additional amino acid sequence which contains secretory or leader sequences, pro-sequences, sequences which aid in purification such as multiple histidine residues, or an additional sequence for stability during recombinant production.

Fragments of the IGS1 polypeptides are also included in the invention. A fragment is a polypeptide having an amino acid sequence that is the same as part of, but not all of, the amino acid sequence of the aforementioned IGS1 polypeptides. As with IGS1 polypeptides, fragments may be “free-standing,” or comprised within a larger polypeptide of which they form a part or region, most preferably as a single continuous region. Representative examples of polypeptide fragments of the invention, include, for example, fragments from about amino acid number 1-20; 21-40, 41-60, 61-80, 81-100; and 101 to the end of IGS1 polypeptide. In this context “about” includes the particularly recited ranges larger or smaller by several, 5, 4, 3, 2 or 1 amino acid at either extreme or at both extremes.

Preferred fragments include, for example, truncation polypeptides having the amino acid sequence of IGS1 polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate receptor activity, including those with a similar activity or an improved activity, or with a decreased undesirable activity. Also included are those that are antigenic or immunogenic in an animal, especially in a human.

Thus, the polypeptides of the invention include polypeptides having an amino acid sequence at least 80% identical to that of SEQ ID NO:2 and/or the polypeptide having the amino acid sequence encoded by the DNA insert contained in the deposit no. CBS 102049 at the Centraalbureau voor Schimmelcultures at Baarn the Netherlands, or fragments thereof with at least 80% identity to the corresponding fragment. Preferably, all of these polypeptide fragments retain the biological activity of the receptor, including antigenic activity. Variants of the defined sequence and fragments also form part of the present invention. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10,1-5, or 1-2 amino acids are substituted, deleted, or added in any combination.

The IGS1 polypeptides of the invention can be prepared in any suitable manner. Such polypeptides include isolated naturally occurring polypeptides, recombinantly produced polypeptides, synthetically produced polypeptides, or polypeptides produced by a combination of these methods. Methods for preparing such polypeptides are well known in the art.

Polynucleotides of the Invention

A further aspect of the invention relates to IGS1 polynucleotides. IGS1 polynucleotides include isolated polynucleotides which encode the IGS1 polypeptides and fragments, and polynucleotides closely related thereto. More specifically, the IGS1 polynucleotide of the invention includes a polynucleotide comprising the nucleotide sequence contained in SEQ ID NO:1, such as the one capable of encoding a IGS1 polypeptide of SEQ ID NO: 2, polynucleotides having the particular sequence of SEQ ID NO: 1 and polynucleotides which essentially correspond to the DNA insert contained in the deposit no. CBS 102049 at the Centraalbureau voor Schimmelcultures at Baarn the Netherlands.

IGS1 polynucleotides further include a polynucleotide comprising a nucleotide sequence that has at least 80% identity over its entire length to a nucleotide sequence encoding the IGS1 polypeptide of SEQ ID NO:2, a polynucleotide comprising a nucleotide sequence that is at least 80% identical to that of SEQ ID NO:1 over its entire length and a polynucleotide which essentially correspond to the DNA insert contained in the deposit no. CBS 102049 at the Centraalbureau voor Schimmelcultures at Baarn the Netherlands.

In this regard, polynucleotides with at least 90% identity are particularly preferred, and those with at least 95% are especially preferred. Furthermore, those with at least 97% are highly preferred and those with at least 98-99% are most highly preferred, with at least 99% being the most preferred. Also included under IGS1 polynucleotides are a nucleotide sequence which has sufficient identity to a nucleotide sequence contained in SEQ ID NO: 1 or to the DNA insert contained in the deposit no. CBS 102049 at the Centraalbureau voor Schimmelcultures at Baarn the Netherlands to hybridize under conditions useable for amplification or for use as a probe or marker. The invention also provides polynucleotides which are complementary to such IGS1 polynucleotides.

IGS1 of the invention is structurally related to other proteins of the G-protein coupled receptor family, as shown by the results of BLAST searches in the public databases. The amino acid sequence of Table 2 (SEQ ID NO:2) has about 30% identity (using BLAST, Altschul S. F. et al. [1997], Nucleic Acids Res. 25:3389-3402) in major parts (amino acid residues 7-222 and 396-470) with the rabbit alpha-1 c adrenergic receptor (Accession # O02824, Miyamoto S. et al, RL Life Sci. (1997) 60:2069-2074), and about 33% identity over amino acid residues 31-220 with the human G-protein coupled receptor RE2 (GenBank Accession # AF091890). The nucleotide sequence of Table 1 (SEQ ID NO:1) is 57% identical to human alpha-1 a/d adrenergic receptor over 266 nucleotide residues (Accession # L31722, Bruno J. F., et al. Biochem. Biophys. Res. Commun. (1991) 179:1485-1490), and 44% identical to the human G-protein coupled receptor RE2 over the first 1426 nucleotide residues (GenBank Accession # AF091890). Furthermore, hydropathy analysis (Hofmann, K., Stoffel, W. (1993) Biol. Chem. Hoppe-Seyler 347:166) of the IGS1-protein sequence indicated the presence of 7 transmembrane domains. Thus, IGS1 polypeptides and polynucleotides of the present invention are expected to have, inter alia, similar biological functions/properties to their homologous polypeptides and polynucleotides, and their utility is obvious to anyone skilled in the art.

Polynucleotides of the invention can be obtained from natural sources such as genomic DNA. In particular, degenerated PCR primers can be designed that encode conserved regions within a particular GPCR gene subfamily. PCR amplification reactions on genomic DNA or cDNA using the degenerate primers will result in the amplification of several members (both known and novel) of the gene family under consideration (the degenerated primers must be located within the same exon, when a genomic template is used). (Libert et al., Science, 1989, 244: 569-572). Polynucleotides of the invention can also be synthesized using well-known and commercially available techniques.

The nucleotide sequence encoding the IGS1 polypeptide of SEQ ID NO:2 may be identical to the polypeptide encoding sequence -contained in SEQ ID NO:1 (nucleotide number 36 to 1559), or it may be a different. nucleotide sequence, which as a result of the redundancy (degeneracy) of the genetic code might also show alterations compared to the polypeptide encoding sequence contained in SEQ ID NO:1, but also encodes the polypeptide of SEQ ID NO:2.

When the polynucleotides of the invention are used for the recombinant production of the IGS1 polypeptide, the polynucleotide may include the coding sequence for the mature polypeptide or a fragment thereof, by itself; the coding sequence for the mature polypeptide or fragment in reading frame with other coding sequences, such as those encoding a leader or secretory sequence, a pre-, or pro- or prepro- protein sequence, or other fusion peptide portions. For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc. Natl. Acad. Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Further preferred embodiments are polynucleotides encoding IGS1 variants comprising the amino acid sequence of the IGS1 polypeptide of SEQ ID NO:2 in which several, 5-10, 1-5, 1-3, 1-2 or 1 amino acid residues are substituted, deleted or added, in any combination.

The polynucleotides of the invention can be engineered using methods generally known in the art in order to alter IGS1-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create amino acid substitutions, create new restriction sites, alter modification (e.g. glycosylation or phosphorylation) patterns, change. codon preference, produce splice variants, and so forth.

The present invention further relates to polynucleotides that hybridize to the herein above-described sequences. In this regard, the present invention especially relates to polynucleotides which hybridize under stringent conditions to the herein above-described polynucleotides. As herein used, the term “stringent conditions” means hybridization will occur only if there is at least 80%, and preferably at least 90%, and more preferably at least 95%, yet even more preferably at least 97%, in particular at least 99% identity between the sequences.

Polynucleotides of the invention, which are identical or sufficiently identical to a nucleotide sequence contained in SEQ ID NO:1 or a fragment thereof, may be used as hybridization probes for cDNA and genomic DNA, to isolate full-length cDNAs and genomic clones encoding IGS1 and to isolate cDNA and genomic clones of other genes (including genes encoding homologs and orthologs from species other than human) that have a high sequence similarity to the IGS1 gene. People skilled in the art are well aware of such hybridization techniques. Typically these nucleotide sequences are 80% identical, preferably 90% identical, more preferably 95% identical to that of the referent. The probes generally will comprise at least 5 nucleotides, and preferably at least 8 nucleotides, and more preferably at least 10 nucleotides, yet even more preferably at least 12 nucleotides, in particular at least 15 nucleotides. Most preferred,.such probes will have at least 30 nucleotides and may have at least 50 nucleotides. Particularly preferred probes will range between 30 and 50 nucleotides.

One embodiment, to obtain a polynucleotide encoding the IGS1 polypeptide, including homologs and orthologs from species other than human, comprises the steps of screening an appropriate library under stringent hybridization conditions with a labeled probe having the SEQ ID NO: 1 or a fragment thereof, and isolating full-length cDNA and genomic clones containing said polynucleotide sequence. Such hybridization techniques are well known to those of skill in the art. Stringent hybridization conditions are as defined above or alternatively conditions under overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA, followed by washing the filters in 0.1×SSC at about 65° C.

The polynucleotides and polypeptides of the present invention may be used as research reagents and materials for discovery of treatments and diagnostics to animal and human disease.

Vectors, Host Cells, Expression

The present invention also relates to vectors which comprise a polynucleotide or polynucleotides of the present invention, and host cells which are genetically engineered with vectors of the invention and to the production of polypeptides of the invention by recombinant techniques. Cell-free translation systems can also be used to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., BASIC METHODS IN MOLECULAR BIOLOGY (1986) and Sambrook et al., MOLECULAR CLONING: A LABORATORY MANUAL, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used. Such systems include, among others, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

For secretion of the translated protein into the lumen of the endoplasmic reticulum, into the periplasmic space or into the extracellular environment, appropriate secretion signals may be incorporated into the desired polypeptide. These signals may be endogenous to the polypeptide or they may be heterologous signals.

If the IGS1 polypeptide is to be expressed for use in screening assays, generally, it is preferred that the polypeptide be produced at the surface of the cell. In this event, the cells may be harvested prior to use in the screening assay. In case the affinity or functional activity of the IGS1 polypeptide is modified by receptor activity modifying proteins (RAMP), coexpression. of the relevant RAMP most likely at the surface of the cell is preferred and often required. Also in this event harvesting of cells expressing the IGS1 polypeptide and the relevant RAMP prior to use in screening assays is required. If the IGS1 polypeptide is secreted into the medium, the medium can be recovered in order to recover and purify the polypeptide; if produced intracellularly, the cells must first be lysed before the polypeptide is recovered.

IGS1 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well-known techniques for refolding proteins may be employed to regenerate active conformation when the polypeptide is denatured during isolation and or purification.

Diagnostic Assays

This invention also relates to the use of IGS1 polynucleotides for use as diagnostic reagents. Detection of a mutated form of the IGS1 gene associated with a dysfunction will provide a diagnostic tool that can add to or define a diagnosis of a disease or susceptibility to a disease which results from under-expression, over-expression or altered expression of IGS1. Also in this event co-expression of relevant receptor activity modifying proteins can be required to obtain diagnostic assays of desired quality. Individuals carrying mutations in the IGS1 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled IGS1 nucleotide sequences. Perfectly matched sequences can be distinguished from mismatched duplexes by RNase digestion or by differences in melting temperatures. DNA sequence differences may also be detected by alterations in .electrophoretic mobility of DNA fragments in gels, with or without denaturing agents, or by direct DNA sequencing. See, e.g., Myers et al., Science (1985) 230:1242. Sequence changes at specific locations may also be revealed by nuclease protection assays, such as RNase and S1 protection or the chemical cleavage method. See Cotton et al., Proc. Natl. Acad. Sci. USA (1985) 85: 4397-4401. In another embodiment, an array of oligonucleotide probes comprising the IGS1 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See for example: M.Chee et al., Science, Vol 274, pp 610-613 (1996)).

The diagnostic assays offer a process for diagnosing or determining a susceptibility to among other things the Diseases as mentioned above, through detection of mutation in the IGS1 gene by the methods described. The diagnostic assays in particular offer a process for diagnosing or determining a susceptibility to psychiatric, and CNS disorders, especially movement disorders, such as tics, tremor, Tourette's syndrome, Parkinson s disease, Huntington's disease, dyskinesias, dystonia and spasms, through detection of mutation in the IGS1 gene by the methods described.

In addition, among other things the Diseases as mentioned above can be diagnosed by methods comprising determining from a sample derived from a subject an abnormally decreased or increased level of the IGS1 polypeptide or IGS1 mRNA. In particular psychiatric and CNS disorders, especially movement disorders, such as tics, tremor, Tourette's syndrome, Parkinson's disease, Huntington's disease, dyskinesias, dystonia and spasms, can be diagnosed by methods comprising determining from a sample derived from a subject an abnormally decreased or increased level of the IGS1 polypeptide or IGS1 mRNA.

Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as an IGS1, in a sample derived from a host are well known to those of skill in the art. Such assay methods include radioimmunoassays, competitive-binding assays, Western Blot analysis and ELISA assays.

In another aspect, the present invention relates to a diagnostic kit for among other things the Diseases or suspectability to one of the Diseases as mentioned above. In particular, the present invention relates to a diagnostic kit for psychiatric and CNS disorders, especially movement disorders, such as tics, tremor, Tourette's syndrome, Parkinson's disease, Huntington's disease, dyskinesias, dystonia and spasms. The kit may comprise:

(a) an IGS1 polynucleotide, preferably the nucleotide sequence of SEQ ID NO:1, or a fragment thereof; and/or

(b) a nucleotide sequence complementary to that of (a); and/or

(c) an IGS1 polypeptide, preferably the polypeptide of SEQ ID NO:2, or a fragment thereof; and/or

(d) an antibody to an IGS1 polypeptide, preferably to the polypeptide of SEQ ID NO: 2; and/or

(e) a RAMP polypeptide required for the relevant biological or antigenic properties of an IGS1 polypeptide.

It will be appreciated that in any such kit, (a), (b), (c) (d) or (e) may comprise a substantial component.

Chromosome Assays

The nucleotide sequences of the present invention are also valuable for chromosome identification. The sequence is specifically targeted to and can hybridize with a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

Antibodies

The polypeptides of the invention or their fragments or analogs thereof, or cells expressing them if required together with relevant RAMP's, may also be used as immunogens to produce antibodies immunospecific for the IGS1 polypeptides. The term “immunospecific” means that the antibodies have substantiall greater affinity for the polypeptides of the invention than their affinity for other related polypeptides in the prior art.

Antibodies generated against the IGS1 polypeptides may be obtained by administering the polypeptides or epitope-bearing fragments, analogs or cells to an animal, preferably a nonhuman, using routine protocols. For preparation of monoclonal antibodies, any technique, which provides antibodies produced by continuous cell line cultures, may be used. Examples include the hybridoma technique (Kohler, G. and Milstein, C., Nature (1975) 256:495497), the trioma technique, the human B-cell hybridoma technique (Kozbor et al., Immunology Today (1983) 4:72) and the EBV-hybridoma technique (Cole et al., MONOCLONAL ANTIBODIES AND CANCER THERAPY, pp. 77-96, Alan R. Liss, Inc., 1985).

The above-described antibodies may be employed to isolate or to identify clones expressing the polypeptide or to purify the polypeptides by affinity chromatography.

Antibodies against IGS1 polypeptides as such, or against IGS1 polypeptide-RAMP complexes, may also be employed to treat among other things the Diseases as mentioned above. In particular, antibodies against IGS1 polypeptides as such, or against IGS1 pplypeptide-RAMP complexes, may be employed to treat psychiatric and CNS disorders, especially movement disorders, such as tics, tremor, Tourette's syndrome, Parkinson's disease, Huntington's disease, dyskinesias, dystonia and spasms.

Animals

Another aspect of the invention relates to non-human animal-based systems which act as models for disorders arising from aberrant expression or activity of IGS1. Non-human animal-based model systems may also be used to further characterize the activity of the IGS1 gene. Such systems may be utilized as part of screening strategies designed to identify compounds which are capable to treat IGS1 based disorders such as among other things the Diseases as mentioned above. In particular, the systems may be utilized as part of screening strategies designed to identify compounds which are capable to treat IGS1 based psychiatric and CNS disorders, especially movement disorders, such as tics, tremor, Tourette's syndrome, Parkinson's disease, Huntington's disease, dyskinesias, dystonia and spasms.

In this way the animal-based models may be used to identify pharmaceutical compounds, therapies and interventions which may be effective in treating disorders of aberrant expression or activity of IGS1. In addition such animal models may be used to determine the LD₅₀ and the ED₅₀ in animal subjects. These data may be used to determine the in vivo efficacy of potential IGS1 disorder treatments.

Animal-based model systems of IGS1 based disorders, based on aberrant IGS1 expression or activity, may include both non-recombinant animals as well as recombinantly engineered transgenic animals.

Animal models for IGS1 disorders may include, for example, genetic models. Animal models exhibiting IGS1 based disorder-like symptoms may be engineered by utilizing, for example, IGS1 sequences such as those described, above, in conjunction with techniques for producing transgenic animals that are well known to persons skilled in the art. For example, IGS1 sequences may be introduced into, and overexpressed and/or misexpressed in, the genome of the animal of interest, or, if endogenous IGS1 sequences are present, they may either be overexpressed, misexpressed, or, alternatively, may be disrupted in order to underexpress or inactivate IGS1 gene expression.

In order to overexpress or misexpress a IGS1 gene sequence, the coding portion of the IGS1 gene sequence may be ligated to a regulatory sequence which is capable of driving high level gene expression or expression in a cell type in which the gene is not normally expressed in the animal type of interest. Such regulatory regions will be well known to those skilled in the art, and may be utilized in the absence of undue experimentation.

For underexpression of an endogenous IGS1 gene sequence, such a sequence may be isolated and engineered such that when reintroduced into the genome of the animal of interest, the endogenous IGS1 gene alleles will be inactivated, or “knocked-out”. Preferably, the engineered IGS1 gene sequence is introduced via gene targeting such that the endogenous IGS1 sequence is disrupted upon integration of the engineered IGS1 gene sequence into the animal's genome.

Animals of any species, including, but not limited to, mice, rats, rabbits, squirrels, guinea-pigs, pigs, micro-pigs, goats, and non-human primates, e.g., baboons, monkeys, and chimpanzees may be used to generate animal models of IGS1 related disorders.

Any technique known in the art may be used to introduce a IGS1 transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus mediated gene transfer into germ lines (van der Putten et al., Proc. Nati. Acad. Sci., USA 82:6148-6152, 1985); gene targeting in embryonic stem cells (Thompson et al., Cell 56:313-321, 1989,); electroporation of embryos (Lo, Mol. Cell. Biol. 3:1803-1B14, 1983); and sperm-mediated gene transfer (Lavitrano et al., Cell 57:717-723, 1989); etc. For a review of such techniques, see Gordon, Transgenic Animals, Intl. Rev. Cytol.115:171-229, 1989.

The present invention provides for transgenic animals that carry the IGS1 transgene in all their cells, as well as animals which carry the transgene in some, but not all their cells, i.e., mosaic animals. (See, for example, techniques described by Jakobovits, Curr. Biol. 4:761-763, 1994) The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M., et al., Proc. Natl. Acad. Sci. USA 89:6232-6236, 1992).

The regulatory sequences required for such a cell-type specific activation will depend upon the particular cell type of interest, and will be apparent to those of skill in the art.

When it is desired that the IGS1 transgene be integrated into the chromosomal site of the endogenous IGS1 gene, gene targeting is preferred. Briefly, when such a technique is to be utilized, vectors containing some nucleotide sequences homologous to the endogenous IGS1 gene of interest (e.g., nucleotide sequences of the mouse IGS1 gene) are designed for the purpose of integrating, via homologous recombination with chromosomal sequences, into and disrupting the function of, the nucleotide sequence of the endogenous IGS1 gene or gene allele. The transgene may also be selectively introduced into a particular cell type, thus inactivating the endogenous gene of interest in only that cell type, by following, for example, the teaching of Gu et al. (Gu, H. et al.-, Science 265:103-106, 1994). The regulatory sequences required for such a cell-type specific inactivation will depend upon the particular cell type of interest, and will be apparent to those of skill in the art.

Once transgenic animals have been generated, the expression of the recombinant IGS1 gene and protein may be assayed utilizing standard techniques. Initial screening may be accomplished by Southern blot analysis or PCR techniques to analyze animal tissues to assay whether integration of the transgene has taken place. The level of mRNA expression of the IGS1 transgene in the tissues of the transgenic animals may also be assessed using techniques which include but are not limited to Northern blot analysis of tissue samples obtained from the animal, in situ hybridization analysis, and RT-PCR. Samples of target gene-expressing tissue, may also be evaluated immunocytochemically using antibodies specific for the target gene transgene product of interest. The IGS1 transgenic animals that express IGS1 gene mRNA or IGS1 transgene peptide (detected immunocytochemically, using antibodies directed against target gene product epitopes) at easily detectable levels may then be further evaluated to identify those animals which display characteristic IGS1 based disorder symptoms.

Once IGS1 transgenic founder animals are produced (i.e., those animals which express IGS1 proteins in cells or tissues of interest, and which, preferably, exhibit symptoms of IGS1 based disorders), they may be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound IGS1 transgenics that express the IGS1 transgene of interest at higher levels because of the effects of additive expression of each IGS1 transgene; crossing of heterozygous transgenic animals to produce animals homozygous for a given integration site in order to both augment expression and eliminate the possible need for screening of animals by DNA analysis; crossing of separate homozygous lines to produce compound heterozygous or homozygous lines; breeding animals to different inbred genetic backgrounds so as to examine effects of modifying alleles on expression of the IGS1 transgene and the development of IGS1-like symptoms. One such approach is to cross the IGS1 transgenic founder animals with a wild type strain to produce an F1 generation that exhibits IGS1 related disorder-like symptoms, such as those described above. The F1 generation may then be inbred in order to develop a homozygous line, if it is found that homozygous target gene transgenic animals are viable.

Vaccines

Another aspect of the invention relates to a method for inducing an immunological response in a mammal which comprises administering to (for example by inoculation) the mammal the IGS1 polypeptide, or a fragment thereof, if required together with a RAMP polypeptide, adequate to produce antibody and/or T cell immune response to protect said animal from among other things one of the Diseases as mentioned above.

Yet another aspect of the invention relates to a method of inducing immunological response in a mammal which comprises delivering the IGS1 polypeptide via a vector directing expression of the IGS1 polynucleotide in vivo in order to induce such an immunological response to produce antibody to protect said animal from diseases.

A further aspect of the invention relates to an immunological/vaccine formulation (composition) which, when introduced into a mammalian host, induces an immunological response in that mammal to an IGS1 polypeptide wherein the composition comprises an IGS1 polypeptide or IGS1 gene. Such immunological/vaccine formulations (compositions) may be either therapeutic immunological/vaccine formulations or prophylactic immunological/vaccine formulations. The vaccine formulation may further comprise a suitable carrier. Since the IGS1 polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Screening Assays

The IGS1 polypeptide of the present invention may be employed in a screening process for compounds which bind the receptor and which activate (agonists) or inhibit activation of (antagonists) the receptor polypeptide of the present invention. Thus,, polypeptides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics.

IGS1 polypeptides are responsible for biological functions, including pathologies. Accordingly, it is desirable to find compounds and drugs which stimulate IGS1 on the one hand and which can inhibit the function of IGS1 on the other hand. In general, agonists are employed for therapeutic and prophylactic purposes for such conditions as among other things the Diseases as mentioned above. In particular, agonists are employed for therapeutic and prophylactic purposes for psychiatric and CNS disorders, especially movement disorders, such as tics, tremor, Tourette's syndrome, Parkinson's disease, Huntington's disease, dyskinesias, dystonia and spasms.

Antagonists may be employed for a variety of therapeutic and prophylactic purposes for such conditions as among other things the Diseases as mentioned above. In particular, antagonists may be employed for a variety of therapeutic and prophylactic purposes for psychiatric and CNS disorders, especially movement disorders, such as tics, tremor, Tourette's syndrome, Parkinson's disease, Huntington's disease, dyskinesias, dystonia and spasms.

In general, such screening procedures involve producing appropriate cells, which express the receptor polypeptide of the present invention on the surface thereof and, if essential coexpression of RAMP's at the surface thereof. Such cells include cells from mammals, yeast, Drosophila or E. coli. Cells expressing the receptor (or cell membrane containing the expressed receptor) are then contacted with a test compound to observe binding, or stimulation or inhibition of a functional response.

One screening technique includes the use of cells which express the receptor of this invention (for example, transfected CHO cells) in a system which measures extracellular pH, intracellular pH, or intracellular calcium changes caused by receptor activation. In this technique, compounds may be contacted with cells expressing the receptor polypeptide of the present invention. A second messenger response, e.g., signal transduction, pH changes, or changes in calcium level, is then measured to determine whether the potential compound activates or inhibits the receptor.

Another method involves screening for receptor inhibitors by determining modulation of a receptor-mediated signal, such as cAMP accumulation and/or adenylate cyclase activity. Such a method involves transfecting an eukaryotic cell with the receptor of this invention to express the receptor on the cell surface. The cell is then exposed to an agonist to the receptor of this invention in the presence of a potential antagonist. If the potential antagonist binds the receptor, and thus inhibits receptor binding, the agonist-mediated signal will be modulated.

Another method for detecting agonists or antagonists for the receptor of the present invention is the yeast-based technology as described in U.S. Pat. No. 5,482,835.

The assays may simply test binding of a candidate compound wherein adherence to the cells bearing the receptor is detected by means of a label directly or indirectly associated with the candidate compound or in an assay involving competition with a labeled competitor. Further, these assays may test whether the candidate compound results in a signal generated by activation of the receptor, using detection systems appropriate to the cells bearing the receptor at their surfaces. Inhibitors of activation are generally assayed in the presence of a known agonist and the effect on activation by the agonist by the presence of the candidate compound is observed.

Further, the assays may simply comprise the steps of mixing a candidate compound with a solution containing an IGS1 polypeptide to form a mixture, measuring the IGS1 activity in the mixture, and comparing the IGS1 activity of the mixture to a standard.

The IGS1 cDNA, protein and antibodies to the protein may also be used to configure assays for detecting the effect of added compounds on the production of IGS1 mRNA and protein in cells. For example, an ELISA may be constructed for measuring secreted or cell associated levels of IGS1 protein using monoclonal and polyclonal antibodies by standard methods known in the art, and this can be used to discover agents which may inhibit or enhance the production of IGS1 (also called antagonist or agonist, respectively) from suitably manipulated cells or tissues. Standard methods for conducting screening assays are well known in the art.

Examples of potential IGS1 antagonists include antibodies or, in some cases, oligonucleotides or proteins which are closely related to the ligand of the IGS1, e.g., a fragment of the ligand, or small molecules which bind to the receptor but do not elicit a response, so-that the activity of the receptor is prevented.

Thus in another aspect, the present invention relates to a screening kit for identifying agonists, antagonists, ligands, receptors, substrates, enzymes, etc. for IGS1 polypeptides; or compounds which decrease or enhance the production of IGS1 polypeptides, which comprises:

(a) an IGS1 polypeptide, preferably that of SEQ ID NO:2;

(b) a recombinant cell expressing an IGS1 polypeptide, preferably that of SEQ ID NO:2;

(c) a cell membrane expressing an IGS1 polypeptide, preferably that of SEQ ID NO:2; or

(d) antibody to an IGS1 polypeptide, preferably that of SEQ ID NO: 2.

It will be appreciated that in any such kit, (a), (b), (c) or (d) may comprise a substantial component.

Prophylactic and Therapeutic Methods

This invention provides methods of treating abnormal conditions related to both an excess of and insufficient amounts of IGS1 activity.

If the activity of IGS1 is in excess, several approaches are available. One approach comprises administering to a subject an inhibitor compound (antagonist) as hereinabove described along with a pharmaceutically acceptable carrier in an amount effective to inhibit activation by blocking binding of ligands to the IGS1, or by inhibiting interaction with a RAMP polypeptide or a second signal, and thereby alleviating the abnormal condition.

In another approach, soluble forms of IGS1 polypeptides still capable of binding the ligand in competition with endogenous IGS1 may be administered. Typical-embodiments of such competitors comprise fragments of the IGS1 polypeptide.

In still another approach, expression of the gene encoding endogenous IGS1 can be inhibited using expression-blocking techniques. Known such techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. USA (1988). Alternatively, oligonucleotides, which form triple helices with the gene, can be supplied. See, for example, Lee et al., Nucleic Acids Res (1979) 6:3073; Cooney et al., Science (1988) 241:456; Dervan et al, Science (1991) 251:1360. These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesized with these or other modified backbones also form part of the present invention.

In addition, expression of the IGS1 polypeptide may be prevented by using ribozymes specific to the IGS1 mRNA sequence. Ribozymes are catalytically active RNAs that can be natural or synthetic (see for example Usman, N, et al., Curr. Opin. Struct. Biol (1996) 6(4), 527-33.) Synthetic ribozymes can be designed to specifically cleave IGS1 mRNAs at selected positions thereby preventing translation of the IGS1 mRNAs into functional polypeptide. Ribozymes may be synthesized with a natural ribose phosphate backbone and natural bases, as normally found in RNA molecules. Alternatively the ribosymes may be synthesized with non-natural backbones to provide protection from ribonuclease degradation, for example, 2′-O-methyl RNA, and may contain modified bases.

For treating abnormal conditions related to an under-expression of IGS1 and its activity, several approaches are also available. One approach comprises administering to a subject a therapeutically effective amount of a compound which activates IGS1, i.e., an agonist as described above, in combination with a pharmaceutically acceptable carrier, to thereby alleviate the abnormal condition. Alternatively, gene therapy may be employed to effect the endogenous production of IGS1 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, Strachan T. and Read A. P., BIOS Scientific Publishers Ltd (1996).

Any of the therapeutic methods described above may be applied to any subject in need of such therapy, including, for example, mammals such as dogs, cats, cows, horses, rabbits, monkeys, and most preferably, humans.

Formulation and Administration

Peptides, such as the soluble form of IGS1 polypeptides, and agonists and antagonist peptides or small molecules, may be formulated in combination with a suitable pharmaceutical carrier. Such formulations comprise a therapeutically effective amount of the polypeptide or compound, and a pharmaceutically, acceptable carrier or excipient. Formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible.

The dosage range required depends on the choice of peptide or compound, the route of administration, the nature of the formulation, the nature of the subject's condition, and the judgment of the attending practitioner. Suitable dosages are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

The following examples are only intended to further illustrate the invention in more detail, and therefore these examples are not deemed to restrict the scope of the invention in any way.

### EXAMPLE 1

### The Cloning of cDNA Encoding a Novel G Protein-Coupled Receptor

### Example 1a

### Homology PCR Cloning of a Genomic Fragment Encoding a Novel G-Protein Coupled Receptor (GPCR)

A PCR based homology cloning strategy was used to isolate partial genomic DNA sequences encoding novel G-protein coupled receptors. The following forward (F11) and reverse (R13) degenerate PCR primers were designed in conserved areas of the neurotensin receptor gene family within transmembrane domain 1 (TM1) and at the boundary of transmembrane domain 3 with intracellular loop n°2 (TM3/I2) respectively:

| | |
|---|---|
| F11 (TM1): | |
| 5′-CATCTTCGTCGTCGGCAC(A, C, G or T)G(C or T)(A, C, G or T)GG(A, C, G or T)AA-3′ | (SEQ ID NO: 3) |
| R13 (TM3/12): | |
| 5′-GGGTGGCAGATGGCCA(A or G)(A or G)(C or T)A(A, C, G or T)C(G or T)(C or T)TC-3′ | (SEQ ID NO: 4) |

In addition a 3′ blocked oligo primer (HNTR1F1STOP) was designed:

| | |
|---|---|
| HNTR1F1STOP: | |
| 5′-ACGGTGGGCAACACGGTGACGGCGTT-3′-3′-dA | (SEQ ID NO: 5) |

The 3′ blocked primer was specific for the human neurotensin receptor (NTR1) cDNA in the TM1 encoding area and partially overlapped (and competed) with the degenerated forward primer. Its 3′-terminus is blocked with a 3′-deoxyadenosine group to prevent polymerase-catalyzed extension (Eurogentec, Belgium catalogue OL-0401-0302).

PCR reactions were carried out in a 60 μl volume and contained 100 ng human genomic DNA (Clontech), 6 μl 10× PCR buffer II (100 mM Tris-HCl pH 8.3; 500 mM KCl, Perkin Elmer), 3.6 μl 25 mM MgCl₂, 0.36 μl dNTPs (25 mM of each dNTP), 1.5 units AmpliTaq™ polymerase (Perkin Elmer), 30 pmoles of each of the degenerated forward and reverse primers and 100 pmoles of the 3′ blocked primer. Reaction tubes were heated at 94° C. for 2 min and then subjected to 20 cycles of denaturation (94° C. 30.sec.), annealing (55° C., 1 min, touchdown −0.25° C./cycle) and extension (72° C., 1min), followed by another 20 cycles of denaturation (94° C,.30 sec.), annealing (50° C., 1 min) and extension (72° C., 1 min). Finally reaction tubes were heated for 5 min at 72° C. PCR reaction products were size fractionated on a 2% agarose gel and stained with ethidium bromide. A fragment of expected size (±300 bp) was purified from gel using the Qiaex-II™ purification kit (Qiagen Inc.) and ligated into the pGEM-T plasmid according to the procedure recommended by the supplier (PGEM-T kit, Promega). The recombinant plasmids thus produced were used to transform competent E. coli SURE™ 2 bacteria (Stratagene).

Transformed cells were plated on LB agar plates containing ampicillin (100 μg/ml), IPTG (0.5 mM) and X-gal (50 μg/ml). Colonies were lifted onto Hybond N+ membranes (Amersham) and DNA was denatured and fixed according to the microwave oven procedure of Buluwela et al. (Nucleic acids Research 17, p452; 1989). Colony lifts were prehybridized at 65° C. for 2 h in modified Church buffer (0.5M phosphate, 7% SDS, 10 mM EDTA) and then hybridised overnight at 65° C. in the same buffer containing 2>10⁶ cpm/ml of an equimolar amount of ³²P-labelled human neurotensin receptor 1 and 2 cDNA probe (NTR1/2). cDNA probes containing the entire coding sequence of human NTR1 and NTR2 were radiolabelled via random primed incorporation of [α-³²P]dCTP to a specific activity of >10⁹ cpm/pg using the Prime-It II kit™ (Stratagene) according to the instructions provided by the supplier. Hybridized filters were washed at high stringency (2×30 min at room temperature in 2×SSC/0.1% SDS followed by 2 washes of 40 min at 65° C. in 0.1×SSC, 0.1% SDS) and autoradiographed overnight. A number of random white colonies that showed no hybridization signal after high stringency washing were selected for DNA sequence analysis.

DNA sequencing reactions were carried out using the ABI Prism™ BigDye Terminator cycle Sequencing reaction kit (PE-ABI). Cycle Sequencing reaction products were purified via EtOH/NaOAc precipitation and loaded on an ABI 373 automated sequencer. Two nearly identical clones (HNT642 and HNT768) were identified that seemed to encode part of a novel member of the GPCR family. We refer to this novel GPCR as IGS1.

TABLE 3
Overview of oligo primers used.

| | |
|---|---|
| SEQ ID NO: 3 | F11: 5′-CATCTTCGTCGTCGGCAC(A, C, G or T)G(C or T)(A, C, G or T)GG(A, |
| | C, G or T)AA-3′ |
| SEQ ID NO: 4 | R13: 5′-GGGTGGCAGATGGCCA(A or G)(A or G)(C or T)A(A, C, G or T)C(G |
| | or T)(C or T)TC-3′ |
| SEQ ID NO: 5 | HNTR1F1STOP: 5′-ACGGTGGGCAACACGGTGACGGCGTT-3′-3′-dA |
| SEQ ID NO: 6 | AP1: 5′-CCATCCTAATACGACTCACTATAGGGC-3′ |
| SEQ ID NO: 7 | AP2: 5′-ACTCACTATAGGGCTCGAGCGGC-3′ |
| SEQ ID NO: 8 | IP11260: 5′-TTTATCTTTAACCTCCTCGTCACCGACC-3′ |
| SEQ ID NO: 9 | IP11261: 5′-TAGTGTTGCAGCGCAAGCCG-3′ |
| SEQ ID NO: 10 | IP11262: 5′-GGCAGCGTTCCACTGACACCAAGACAATGG-3′ |
| SEQ ID NO: 11 | IP11263: 5′-CAGCGTTCCACTGACACCAAGACAATGG-3′ |
| SEQ ID NO: 12 | IP11264: 5′-AAGGCGAACAGGTGGGTGAGGCTAACC-3′ |
| SEQ ID NO: 13 | IP11515: 5′-TGGCGAAGGCGAACAGGTGG-3′ |
| SEQ ID NO: 14 | IP11516: 5′-GCGAAGGCGAACAGGTGGGTGAGG-3′ |
| SEQ ID NO: 15 | IP11684: 5′-CTAGTGTTGCAGCGCAAGCCGCAG-3′ |
| SEQ ID NO: 16 | IP12261: 5′-CACAGAAAGCATAACCAGTGATTGAACC-3′ |
| SEQ ID NO: 17 | IP12262: 5′-GCTTTAGGTTCCTGGAATCCCATTTGG-3′ |
| SEQ ID NO: 18 | IP12264: 5′-TTGTCACCAGCATAGGCACTGAGTG-3′ |

### Example 1b

### Cloning of cDNA Fragments Containing the Complete IGS1 Coding Sequence

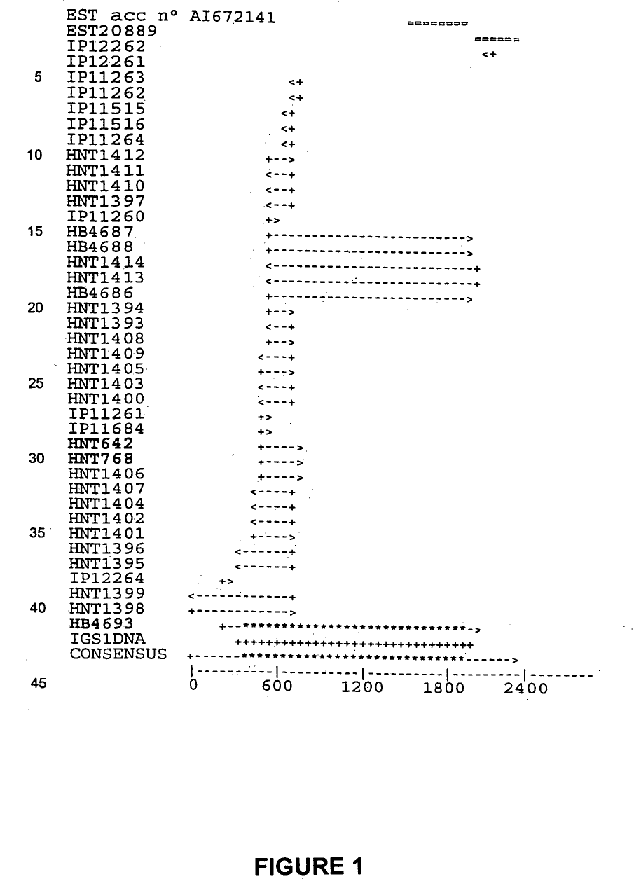

The complete coding sequence of IGS1 cDNA was obtained via rapid amplification of cDNA ends (RACE analysis). 5′- and 3′ RACE PCRs were performed on Marathon-Ready™ human brain cDNA (Clontech n° 7400-1), using the adaptor primer 1 (AP1: SEQ ID NO: 6) provided with the Marathon™ cDNA amplification kit (Clontech K1802-1) and IGS1 specific primers IP11261 (3′ RACE; SEQ ID NO: 9) and IP11262 and IP11263 (5′ RACE; SEQ ID NO: 10 and 11 respectively), based on the DNA sequence of clones HNT642 and HNT768 (FIG. 1). Subsequently a nested RACE PCR was carried out with adaptor primer 2 (AP2; SEQ ID NO: 7) and the IGS1 specific nested primers IP11260 (3′ RACE; SEQ ID NO: 8) and IP11515 and IP11516 (5′RACE; SEQ ID NO: 13 and 14 respectively)). Primary and nested PCR RACE reactions were performed according to the instructions of the Marathon-Ready™ cDNA user manual provided by Clontech. The nested PCR RACE products were separated on a 1% agarose gel and stained with EtBr. The gel was blotted onto Hybond N⁺ membranes and hybridized overnight in Church hybridisation buffer at 65° C. with the ³²P-labelled insert of clone HNT642.

Southern blot analysis of both the AP2/IP11515 and AP2/IP11516 5′RACE nested PCR reactions showed several positive bands (±200 bp, ±250 bp, ±330 bp, ±360 bp, ±400 bp and ±700 bp). Each of these bands was purified from gel and cloned in the pGEM-T plasmid vector (the respective PCR fragments from the IP11515 and IP11516 nested 5′ RACE reactions were pooled before cloning). 3-4 random colonies from each fragment were sequenced (=clones HNT1393-1412) (FIG. 1).

The nested AP-2/IP11260 3′-RACE PCR reaction showed several bands. The largest 3′ nested RACE PCR fragment (±1,550 bp) that hybridized with the IGS1 probe was purified from gel, ligated in pGEM-T (Promega) and used to transform competent E. coli SURE II cells. IGS1 specific transformants from this ligation reaction were identified after colony hybridization using the ³²P-labelled insert of clone HNT642. The colony blots were hybridized to the probe as specified before and washed at high stringency (0.1×SSC 0.1% SDS at 65° C. for 30 min). The hybridization screening of the 3′ RACE nested PCR library yielded 3 positive clones. Two of these (HNT 1413-1414) were sequenced.

In two additional experiments, three more 3′ RACE cDNA clones (HB4686, HB4687 and HB4688) were obtained from Marathon-Ready™ human brain cDNA (Clontech), following the procedures outlined in the manual provided by the supplier (Clontech PT1156-1). In one experiment products from a primary 3′ RACE reaction (obtained using IGS1 specific primer IP11261 and adaptor primer AP1) were reamplified using the hemi-hested primer pair IP11260/IP12261 (SEQ ID NO: 8 and 16 respectively). This yielded an expected ±1400 bp fragment which was purified from gel and cloned in the pGEM-T plasmid vector, yielding clones HB4686 and HB4687. In the other experiment primary 3′ RACE PCR products (obtained using IGS1 specific primer IP11684 (SEQ ID NO: 15) and adaptor primer API) were reamplifed using the nested primer pair IP11260/IP12261. A ±1400 bp fragment resulting from this reaction was purified and cloned into the pGEM-T plasmid vector, yielding clone HB4688.

All IGS1 cDNA clones that were isolated were fully sequenced and could be assembled into a single contig (FIG. 1). The part of this contiguous cDNA sequence that was determined from at least four independent cDNA clones is presented here as IGS1 DNA (SEQ ID NO: 1) Translation of this contig revealed a long open reading frame predicting a protein of 508 amino acids which showed good homology to GPCR proteins (1GS1 PROT; SEQ ID NO: 2). A computer-assisted homology search (Blastn; Altschul S. F. et al. [1997], Nucleic Acids Res. 25:3389-3402) of the DNA sequence of the IGS1 contig against the expressed sequence tag database (dbest) showed the presence of EST20889 (accession no M318717) and EST accession no A1672141 which both overlapped with the 3′ end of the IGS1 contig but were outside of the IGS1 open reading frame (FIG. 1).

### Example 1c

### Isolation of a Contiguous cDNA Fragment Containing the Complete IGS1 Coding Sequence

A contiguous IGS1 cDNA clone was generated via overlap-PCR on the clone HNT1398 and HNT1413 templates. 100 ng of HNT1398 plasmid DNA and 10 ng HNT1413 plasmid were PCR amplified in separate reactions (50 μl) using primer pairs IP12264/IP11264 (SEQ ID NO: 18 and 12 respectively) and IP11260/IP12262 (SEQ ID NO: 8 and 17 respectively) respectively, (30 PCR cycles of denaturation [94° C., 30 sec.], annealing [60° C., 30 sec.] and extension [72° C., 1 min] using the Expand™ High Fidelity PCR system [Boehringer]). One μl amounts of each PCR reaction were combined and reamplified using primer pair IP12264/IP12261 under the same conditions. This overlap-PCR reaction yielded a band of ±1730 bp, which was purified from gel and ligated into the pGEM-T plasmid vector. Recombinant plasmids were used to transform competent E. coli DH5αF′ bacteria. Transformed cells were plated on LB agar plates containing ampicillin (100 μg/ml). Plasmid DNA was prepared from a number of random colonies and the insert size was determined via restriction digestion. Three clones containing a ±1730 bp insert were sequenced. The sequence of clone HB4693 was completely identical to that of the consensus IGS1 cDNA sequence (see FIG. 1). The bacterial strain harboring plasmid HB4693 was recloned after replating on LB agar plates containing 100 μg ampicillin/ml and deposited both in the Innogenetics strain list (ICCG #4297) and at the Centraalbureau voor Schimmelculturen (CBS) in Baarn, The Netherlands (deposit no. CBS 102049). Plasmid DNA prepared from the recloned isolate was resequenced and found to be identical to the consensus sequence determined previously.

Note: we later found out that the primer IP12262 sequence was not included in the insert sequence of clone HNT1413 and that as a consequence no amplicon could have been generated from the HNT1413 template. Therefore we assume that the successful amplification of an overlap fragment occurred via direct overlap between the HNT1413 plasmid DNA (carried over into the overlap PCR reaction tube) and the amplicon generated from the HNT1398 template.

### EXAMPLE 2

### Northern and “MTE Array” Analysis of IGSI

### Example 2a

### Construction of the pcDNA3.1(+)hu IGSI Expression Vector

5 μg pcDNA3.1(+) (Invitrogen) was cut with HindIII (3 h 37° C.), blunted with T4 polymerase in the presence of dNTP's (0.25 mM f.c.), and analyzed on gel. The linearized DNA was eluted from gel (using the Qiaex II extraction kit, Qiagen) and dissolved in 40 μl H₂O. This DNA was digested with NotI, and again analyzed on gel. The obtained 5364 bp vector fragment was eluted from gel using the QiaexII gel extraction kit and dissolved in 40 μl H₂O. 5 μl was analyzed on gel to check size, quantity and purity.

The human IGS1 coding sequence was obtained after NaeI/NotI digestion (3 h, 37° C.) of 5 μg pGEM-ThuIGS1 plasmid (ICCG #4297). The digestion resulted in 3 fragments of 400 bp, 1629 bp and 2702 bp as shown by agarose gel electrophoresis. The 1629 bp fragment was eluted from gel (QiaexII) and redissolved in 40 μl H₂O. 5 μl was analyzed on gel.

One μl of the HindIII digested pcDNA3.1(+) vector, 3 μl insert and 16 μl H₂O were added to a Ready-To-Go ligase tube (T4 DNA ligase, Amersham Pharmacia Biotech) and incubated for 1 h at RT. Two μl of the ligation mix was used to transform chemically competent DH5αF′ bacteria. 200 μl of the transformed bacteria were plated on LB plates (100 μg ampicillin/ml) and grown overnight at 37° C. 16 random colonies were picked and cultured in 3 ml LB medium containing ampicillin. Plasmid DNA was prepared using the BioRobot™ 9600 nucleic acid purification system (Qiagen) and analyzed via restriction analysis using the NotI, PstI and SphI restriction enzymes. DNA from one colony with the correct restriction pattern was partially sequenced to verify the insertion points and found to have the expected sequence. The partially sequenced colony was deposited in the Innogenetics strainlist (ICCG #4350) and a large amount of DNA (MegaPrep, Qiagen 500 kit) was prepared from the deposited strain. Sequence analysis of this large scale DNA prep (500 μl of 3 μg/μl) confirmed the expected sequence.

### Example 2b

### MTE (Multiple Tissue Expression) Array Analysis

25 ng human IGS1 DNA (1093 bp AatII insert from pcDNA3.1.huIGS1 [ICCG #4350]) was labelled using (α-³²P)-dCTP. The labeled probe was purified using a Micro Bio-Spin P-30 column (BioRad). 16×10⁶ cpm labelled huIGS1 cDNA probe was mixed with 30 μg of C₀t-1 DNA, 150 μg of sheared herring sperm DNA and 50 μl 20×SSC in a total volume of 200 μl, heated for 5 min. at 95° C. and then incubated for 30 min. at 68° C. This mixture was added to 5 ml Express Hyb solution and evenly distributed over the human Multiple Tissue Expression (MTE) array (Clontech #7775-1). The array was hybridized overnight at 68° C. The blot was rinsed four times for 20 min at 65° C. in 2×SSC/1% SDS and two times for 20 min at 55° C. in 0.1×SSC/0.5% SDS. The blot was autoradiographed using X-ray film.

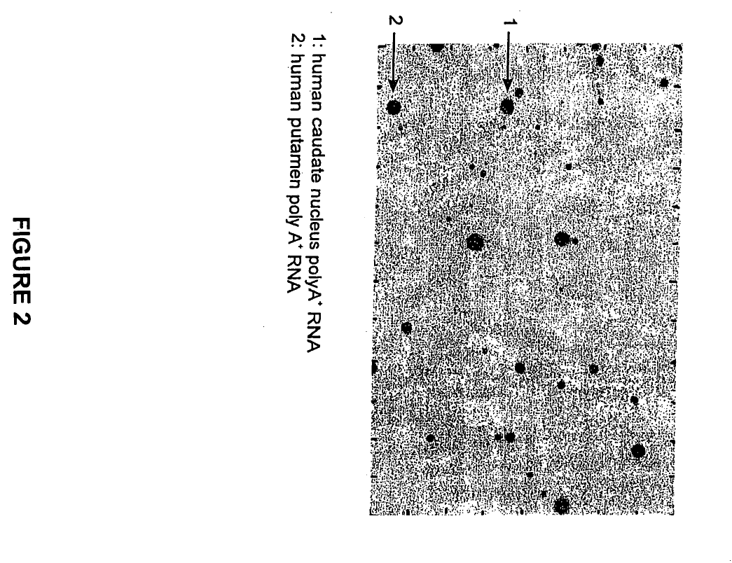

Hybridization of the IGS1 probe on the MTE array, showed strong signals on caudate nucleus and putamen only (FIG. 2).

### Example 2c

### Northern Blot Analysis

25 ng human IGS1 DNA (1093 bp AatII insert from pcDNA3.1.huIGS1 [ICCG #4350]) was labelled using (α-³²P)-dCTP. The labeled probe was purified using a Micro Bio-Spin P-30 column (BioRad). 8×10⁶ cpm labelled huIGS1 cDNA probe was denatured for 5 min. at 95° C. and added to 5 ml Express Hyb solution and evenly distributed over the Human Brain MTN Blots II or IV (Clontech #7755-1 and #7769-1 respectively). The blot was hybridized overnight at 68° C. The blot was rinsed four times for 10 min at room temperature in 2×SSC/0.05% SDS and two times for 40 min at 50° C. in 0.1×SSC/0.1% SDS. The blot was autoradiographed using X-ray film.

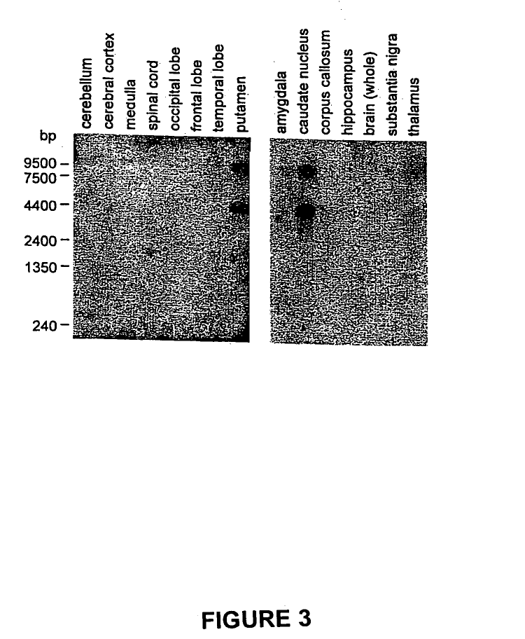

Hybridization of the IGS1 probe on Northern blots of RNA from different human brain regions showed 2 strong bands of approximately 4,400 and 9,000 nucleotides (nt) in both putamen and caudate nucleus (FIG. 3). This lower band was slightly more intense in caudate nucleus, while the reverse was the case for putamen. The 4,400 and 9,000 nt bands could also be seen in thalamus but both were very weak. In addition a very faint 9,000 nt transcript was detected in substantial nigra but no 4,400 band. Finally extremely weak 9,000 nt bands were observed in cerebellum, medulla and amygdala. The 4,400 nt band could not be observed in thalamus and substantia nigra. These results are in agreement with the results of the MTE analysis, in the sense that the strongest expression of IGS1 was observed in caudate nucleus and in putamen. However the presence of 2 transcripts is unexpected. Whereas the. 4,400 nt band most likely corresponds to the IGS1 mRNA, the origin of the 9,000 nt band is unclear. Since the IGS1 gene does not contain introns (at least not within the coding area) the 9,000 nt transcript is probably not due to an unspliced or alternatively spliced transcript. It might be a IGS1 transcript with an alternative poly-adenylation site or else it is just a cross-hybridizing species. We assume that in cases where only a very weak 9,000 nt transcript was detected and no 4,400 transcript, this is due to the fact that the 9,000 nt transcript is slightly more intense than the 4,400 transcript and that this lower band therefore was just below the detection limit of the Northern assay.

These results were confirmed by in situ hybridization analysis of IGS1 in rat brain, in which IGS1 expression was detected in anatomically identical areas as described above.

### EXAMPLE 3

### Screening of Putative Ligands for IGS1

### Example 3a

### Construction of IGS-1 Transfected CHOG 16-Cells

To identify ligands for IGS1, Chinese Hamster Ovary (CHO) cells were stably transfected with IGS1. Since the G-protein coupling mechanism of IGS1 was unknown, a specific CHO-cell strain was used, which expresses the G-protein G 16 (CHOG 16, Molecular Devices), known as “universal adapter” for GPCRs (Milligan G. et al. (1996) Trends Pharmacol. Sci. 17: 235-7).

The Materials used included: IGS1-pREP9 vector; SuperFect Transfection Reagent (Qiagen); Growth-medium: CHO-S-SFM II (Gibco BRL), supplemented with 10% FCS, 2 mM L-glutamin, Hygromycin B 400 μg/ml; Selection-medium: CHO-S-SFM II (Gibco BRL), supplemented with 10% FCS, 2 mM L-glutamin, Hygromycin B 400 μg/ml and Geneticin 500 μg/ml; RNeasy Mini Kit (Qiagen), DNase I (Ambion, 2 U/μl), SuperScript II (Gibco BRL), SuperScript-II 200 U (Gibco BRL), AmpliTaq (PerkinElmer)

The IGS1 coding sequence was cloned from pcDNA3.1.huIGS1 [ICCG # 4350] into pREP9 (Invitrogen) via XhoI/NheI sites. CHOGα16 cells were transfected with SuperFect (Qiagen), as described by the manufacturer. Transfections were done in T25 flasks. After 24 hours in Growth-medium, medium was removed and replaced by Selection-medium. After growing to confluency in Selection-medium the polyclonals were passed two times in T75 flasks. To obtain monoclonals, cells were seeded in Limited Dilution.

Selection of monoclonals was done by RT-PCR. 14 monoclonals were tested. RNA was isolated from monoclonals (1 confluent well from 24 wells plate) with the RNeasy Mini Kit (Qiagen),, according to the supplied protocol. RNA was treated with DNase I (Ambion, 2 U/μl), 1 U per sample. Half of the RNA sample was used for RT-PCR using SuperScript II (Gibco BRL). Primer annealing was carried out with RNA and oligo-dT16 (0.6 μM) for 10 min at 65° C. to 15° C. First Strand Buffer (Gibco BRL) with dNTP's 0.43 mM each, DTT 10 mM, 20 U RNasin (Promega, 40 U/μl) and SuperScript II 200 U (Gibco BRL, 200 U/μl) to a final volume of 3.0 μl was added, followed by incubation at 42° C. for 1 hour.

PCR was carried out in 25 μl with IGS1 specific internal primers, with AmpliTaq (PerkinElmer). Firstly, PCR with 35 cycles was performed. To confirm the positive monoclonals and to select the best ones, another PCR with fewer cycles and higher annealing temperature was performed. Per PCR reaction 2 μl First Strand cDNA (from 30 μl) was used.

The six best monoclonals were grown in T75 flask to confluency and frozen in growth medium, containing 10% DMSO.

### Example 3b

### Intracellular Calcium Measurements

The CHOG 16-IGS1 cells were functionally screened on a Fluorometric Imaging Plate Reader (FLIPR) to-measure mobilisation of intracellular calcium in response to putative ligands.

For cell preparation, the following materials were used: clear, flat-bottom, black well 96-well plates (Costar); Growth-medium: Nut-Mix F-12 (HAM) with Glutamax (Gibco) supplemented with 10% fetal calf serum (Gibco); incubator: 5% CO2, 37° C. (Nuaire)

Cells were seeded 24 hours or 48 hours prior to the experiment into black wall microplates. The cell density was 0.8×10⁻⁴ cells/well for 48 hour incubation and 2.2×10⁻⁴ cells/well for 24 hour incubation. All steps were done under sterile conditions.

For dye loading, the following materials were used: 2 mM dye stock: 1 mg Fluo-4 (Molecular Probes) solubilized in 443 μl low-water DMSO (Sigma) (aliquots were stored at −20); 20% pluronic acid solution: 400 mg pluronic acid (Sigma) solubilized in 2 ml low-water DMSO (Sigma) at 37° C. (stored at room temperature); Dye/pluronic acid mixture: immediately before use, equal volumes of the dye stock and 20% pluronic acid were mixed (the dye and pluronic acid had a final concentration of 1 mM and 10%, respectively); Probenicid, 250 mM stock solution: 710 mg probenicid (Sigma) solubilized in 5 ml 1N NaOH and mixed with 5 ml Hank's BSS without phenol red (Gibco) supplemented with 20 mM HEPES; Loading-buffer: 10.5 ml Hank's BSS without phenol red (Gibco) supplemented with 20 mM HEPES, 105 μl probenicid, 210 μl 1M HEPES; Wash-buffer: Hank's BSS without phenol red (Gibco) supplemented with 20 mM HEPES (Gibco) and 2.5 mM probenicid.

The 2 mM dye stock was mixed with an equal volume of 20% (w/v) pluronic acid immediately before adding to the Loading-buffer. The Growth-medium was aspirated out of the well without disturbing the confluent cell layer. 100 μl Loading-buffer was dispensed into each well using a Multidrop (Labsystems). Cells were incubated in a 5% CO2, 37° C. incubator for 30 minutes. In order to calculate the background fluorescence, some wells were not dye loaded. After dye loading, cells were washed three times with Wash-buffer (automated Denley cell washer) to reduce the basal fluorescence to 20.000-25.000 counts above background. 100 μl Wash-buffer was added and cells were incubated at 37° C. till the start of the experiment.

Compounds to be screened were diluted in Hank's BSS without phenol red (Gibco) supplemented with 20 mM HEPES (Gibco) and 0.1% BSA (Sigma). Intracellular calcium detection with FLIPR was carried out as described by the manufacturer (Molecular Devices). The FLIPR setup parameters were set to 0.4 sec exposure length, filter 1, 50 μl fluid addition, pipettor height at 125 μl, Dispense Speed 40 μl/sec without mixing.

1-18. (CANCELED)

19. A method of identifying agonists to the IGS1 polypeptide comprising an amino acid sequence that is at least 80% identical to the amino acid sequence of SEQ ID NO: 2 over its entire length, comprising:
(a) contacting a cell which produces a IGS1 polypeptide with a test compound; and
(b) determining whether the test compound effects a signal generated by activation of the IGS1 polypeptide.

20. An agonist identified by the method of claim 19.

21. The method for identifying antagonists to the IGS1 polypeptide comprising an amino acid sequence that is at least 80% identical to the amino acid sequence of SEQ ID NO: 2 over its entire length, comprising:
(a) contacting a cell which produces a IGS1 polypeptide with an agonist; and
(b) determining whether the signal generated by said agonist is diminished in the presence of a candidate compound.

22. An antagonist identified by the method of claim 21.

23-24. (CANCELED)

25. The method of claim 19 wherein the IGS1 polypeptide comprises an amino acid sequence identical to the amino acid sequence of SEQ ID NO: 2.

26. The method of claim 21 wherein the IGS1 polypeptide comprises an amino acid sequence identical to the amino acid sequence of SEQ ID NO: 2.


SEQUENCE LISTING (18 sequences)

SEQ ID NO: 1 - DNA, 1659 bp, Homo sapiens
  CDS (36)..(1559)
gcctgcaacctgtcycacgccctctggctgttgccatgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcttggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatggcatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttgaagttagttctaaggcaaaccttgaaaatcagtccttcagccacagctatttagagctttaaaactaccaggttcaatcactggttatgctttctgtg

SEQ ID NO: 2 - protein, 508 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVLVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDGMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 3 - DNA, 27 bp, Artificial Sequence
  Description of Artificial SequencePrimer
catcttcgtcgtcggcacngynggnaa

SEQ ID NO: 4 - DNA, 26 bp, Artificial Sequence
  Description of Artificial SequencePrimer
gggtggcagatggccarryanckytc

SEQ ID NO: 5 - DNA, 27 bp, Artificial Sequence
  Description of Artificial SequencePrimer
acggtgggcaacacggtgacggcgtta

SEQ ID NO: 6 - DNA, 27 bp, Artificial Sequence
  Description of Artificial SequencePrimer
ccatcctaatacgactcactatagggc

SEQ ID NO: 7 - DNA, 23 bp, Artificial Sequence
  Description of Artificial SequencePrimer
actcactatagggctcgagcggc

SEQ ID NO: 8 - DNA, 28 bp, Artificial Sequence
  Description of Artificial SequencePrimer
tttatctttaacctcctcgtcaccgacc

SEQ ID NO: 9 - DNA, 20 bp, Artificial Sequence
  Description of Artificial SequencePrimer
tagtgttgcagcgcaagccg

SEQ ID NO: 10 - DNA, 30 bp, Artificial Sequence
  Description of Artificial SequencePrimer
ggcagcgttccactgacaccaagacaatgg

SEQ ID NO: 11 - DNA, 28 bp, Artificial Sequence
  Description of Artificial SequencePrimer
cagcgttccactgacaccaagacaatgg

SEQ ID NO: 12 - DNA, 27 bp, Artificial Sequence
  Description of Artificial SequencePrimer
aaggcgaacaggtgggtgaggctaacc

SEQ ID NO: 13 - DNA, 20 bp, Artificial Sequence
  Description of Artificial SequencePrimer
tggcgaaggcgaacaggtgg

SEQ ID NO: 14 - DNA, 24 bp, Artificial Sequence
  Description of Artificial SequencePrimer
gcgaaggcgaacaggtgggtgagg

SEQ ID NO: 15 - DNA, 24 bp, Artificial Sequence
  Description of Artificial SequencePrimer
ctagtgttgcagcgcaagccgcag

SEQ ID NO: 16 - DNA, 28 bp, Artificial Sequence
  Description of Artificial SequencePrimer
cacagaaagcataaccagtgattgaacc

SEQ ID NO: 17 - DNA, 27 bp, Artificial Sequence
  Description of Artificial SequencePrimer
gctttaggttcctggaatcccatttgg

SEQ ID NO: 18 - DNA, 25 bp, Artificial Sequence
  Description of Artificial SequencePrimer
ttgtcaccagcataggcactgagtg